(19) United States
(12) Patent Application Publication
(10) Pub. No.: US 2026/0207155 A1
(43) Pub. Date: Jul. 23, 2026

(54) STORAGE MEDIUM, IMAGING CONTROL APPARATUS, AND MEDICAL SYSTEM

(71) Applicant: Konica Minolta, Inc., Tokyo (JP)

(72) Inventors: Takeshi Yatsuzuka, Tokyo (JP); Takuya Ishii, Tokyo (JP); Satoshi Hasegawa, Tokyo (JP)

(73) Assignee: Konica Minolta, Inc., Tokyo (JP)

(21) Appl. No.: 19/457,718

(22) Filed: Jan. 23, 2026

(30) Foreign Application Priority Data
Jan. 23, 2025 (JP) 2025-009760

(51) Int. Cl. A61B 6/00; A61B 6/46; G06T 7/00; G16H 50/20

(52) U.S. Cl. CPC A61B 6/5217; A61B 6/463; A61B 6/54; A61B 6/56; G06T 7/0012; G16H 50/20; G06T 2207/30096

(57) ABSTRACT

A non-transitory computer-readable storage medium stores a program causing a computer to acquire a medical image in which a subject is imaged, acquire diagnosis support information generated by computer processing on the medical image, determine to transmit the acquired medical image to an external apparatus, and display the medical image and the diagnosis support information before the determining.

### CROSS-REFERENCE TO RELATED APPLICATION

The entire disclosure of Japanese Patent Application No. 2025-009760, filed on Jan. 23, 2025, including description, claims, drawings and abstract is incorporated herein by reference.

### BACKGROUND OF THE INVENTION

### Technical Field

The present disclosure relates to a storage medium, an imaging control apparatus, and a medical system.

### Description of Related Art

A computer diagnosis support apparatus infers presence or absence of a lesion and an abnormal part in a medical image acquired from an imaging apparatus, and presents the diagnosis support information to a doctor, thereby supporting the diagnosis of an X-ray interpreter or the like. In addition, in recent years, an STAT image report in which the radiological technologist conveys a finding of a captured medical image to an X-ray interpreter or the like has started to attract attention. Japanese Unexamined Patent Publication No. 2003-123456 describes an information processing apparatus that controls output of diagnosis support information on a medical image by an inference section to an external apparatus on the basis of an image inspection result of an acquired medical image.

### SUMMARY OF THE INVENTION

However, the conventional technology is not configured to provide a radiographer with diagnosis support information before a process of determining whether an image inspection result of a medical image is pass or fail. In this case, for the radiological technologist or the like to check diagnosis support information, he/she needs to wait for the result of a pass determination process on an image inspection result. Therefore, in a case where a medical image includes a lesion with a high degree of urgency such as pneumothorax, there is a problem in that the radiological technologist cannot promptly inform a doctor of the findings in the image.

Therefore, in order to solve the problem described above, an object of the present disclosure is to provide a storage medium storing a program, an imaging control apparatus, and a medical system that allows the radiological technologist to quickly grasp a lesion or the like in a medical image.

To achieve at least one of the abovementioned objects, according to an aspect of the present disclosure, a non-transitory computer-readable storage medium reflecting one aspect of the present disclosure stores a program causing a computer to:
acquire a medical image in which a subject is imaged;
acquire diagnosis support information generated by computer processing on the medical image;
determine to transmit the acquired medical image to an external apparatus; and
display the medical image and the diagnosis support information before the determining.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 shows an example of a schematic configuration of an image capturing system according to a first embodiment;

FIG. 2 is a flowchart showing an example of the operation of an imaging control apparatus in a case where a medical image is transmitted to an image inspection apparatus according to the first embodiment;

FIG. 3 shows a medical image and diagnosis support information displayed on an imaging screen of a display part according to the first embodiment;

FIG. 4 is a flowchart showing an example of the operation of the image inspection apparatus in a case where it is determined whether an image inspection result of a medical image is pass according to the first embodiment;

FIG. 5 shows a medical image and diagnosis support information displayed on an image inspection screen of the display part according to the first embodiment;

FIG. 6 shows an example of a schematic configuration of an image capturing system according to a second embodiment;

FIG. 7 is a flowchart showing an example of the operation of the imaging control apparatus in a case where a medical image is transmitted to an image management apparatus according to the second embodiment; and

FIG. 8 shows a medical image and diagnosis support information displayed on the imaging screen of the display part according to the second embodiment.

### DETAILED DESCRIPTION

Hereinafter, one or more embodiments of the present disclosure will be described with reference to the drawings. However, the scope of the invention is not limited to the disclosed embodiments.

Hereinafter, with reference to the accompanying drawings, a program, an imaging control apparatus, and a medical system according to a preferred embodiment(s) of the present disclosure will be described in detail.

### First Embodiment

### Configuration Example of Image Capturing System 100A

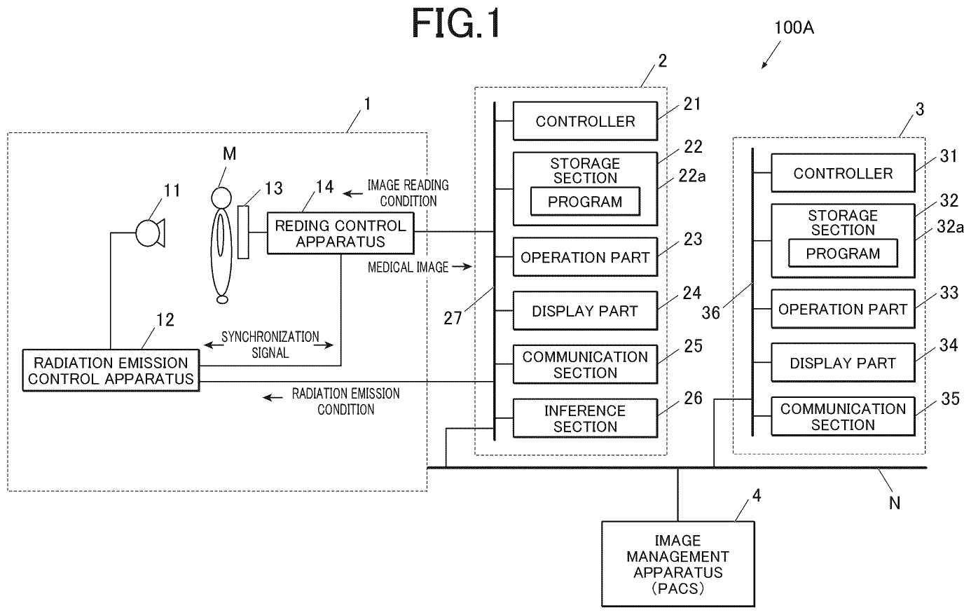

FIG. 1 shows an example of a schematic configuration of an image capturing system 100A according to a first embodiment. The image system 100A corresponds to an example of a medical system and includes an imaging apparatus 1, an imaging control apparatus 2, an image inspection apparatus 3, an image management apparatus 4, and the like. The imaging apparatus 1, the imaging control apparatus 2, the image inspection apparatus 3, and the image management apparatus 4 are communicably connected to each other via a network N such as a LAN, the Internet, or a WAN. A communication method of the network N may be wired communication or wireless communication. LAN is an abbreviation for Local Area Network. WAN is an abbreviation for Wide Area Network.

The imaging apparatus 1 captures a medical image of a predetermined imaging part of a subject M. The imaging control apparatus 2 controls radiographic imaging that is performed by the imaging apparatus 1 and also controls a reading operation of a radiographic image that is performed by the imaging apparatus 1. The image inspection apparatus 3 determines whether an image inspection result of a medical image acquired from the imaging control apparatus 2 via the network N is pass (acceptable). The image management apparatus 4 is also called PACS, and stores and manages medical images and the like received from the imaging control apparatus 2, the image inspection apparatus 3, and the like via the network N. PACS is an abbreviation for Picture Archiving and Communication System. Each apparatus constituting the image capturing system 100A complies with the DICOM standard, and communication between the apparatuses is performed in accordance with to the DICOM standard. DICOM is an abbreviation for Digital Image and Communications in Medicine.

### Configuration Example of Imaging Apparatus 1

Next, the imaging apparatus 1 will be described in detail. As illustrated in FIG. 1, the imaging apparatus 1 includes a radiation source 11, a radiation emission control apparatus 12, a radiation detector 13, and a reading control apparatus 14. The radiation emission control apparatus 12 and the reading control apparatus 14 are connected to each other via a communication cable or the like, and exchange synchronization signals with each other, thereby synchronizing a radiation emission operation and an image reading operation. In FIG. 1, the radiation detector 13 and the reading control apparatus 14 are separately configured, but the radiation detector 13 and the reading control apparatus 14 may be integrally configured.

The radiation source 11 is arranged at a position opposite to the radiation detector 13 with the subject M interposed therebetween. The radiation source 11 irradiates the subject M with radiation such as X-rays under the control of the radiation emission control apparatus 12. The radiation emission control apparatus 12 performs radiographic imaging by controlling the radiation source 11 based on radiation emission conditions input from the imaging control apparatus 2. The radiation emission conditions include, for example, a pulse rate, a pulse width, a pulse interval, the number of imaging frames per imaging, a value of X-ray tube current, a value of X-ray tube voltage and a type of additional filter. The pulse rate is the number of times that radiation is emitted per second, and matches a frame rate described below. The pulse width is a radiation emission time per radiation emission. The pulse interval is a period of time from the start of one radiation emission to the start of the next radiation emission, and matches a frame interval described below.

The radiation detector 13 is constituted of, for example, a semiconductor image sensor such as an FPD. FPD is an abbreviation for Flat Panel Detector. The FPD includes a substrate formed of glass or the like. At predetermined positions on the substrate, detection elements including pixels are arranged in a matrix. The detection elements detect the radiation having been emitted from the radiation source 11 and having passed through at least the subject M according to the intensity of the radiation, convert the detected radiation into an electrical signal, and accumulate the electrical signal. Each pixel includes a switching part such as a TFT. TFT is an abbreviation for Thin Film Transistor. Examples of the type of FPD include an indirect conversion type and a direct conversion type, and either type may be used. The indirect conversion type is a system in which the radiation is converted into the electrical signal by a photoelectric conversion element via a scintillator. The direct conversion type is a system of directly converting the radiation into the electrical signal.

The reading control apparatus 14 controls the switching part of each pixel of the radiation detector 13 based on image reading conditions input from the imaging control apparatus 2 or the like. The reading control apparatus 14 switches reading of the electrical signal accumulated in each pixel of the radiation detector 13, and acquires image data by reading the electrical signal accumulated in the radiation detector 13. The image data is each frame image of a dynamic image or a still image. In the present embodiment, the still image and the dynamic image are referred to as medical images. If a structure exists between the radiation source 11 and the radiation detector 13, the amount of radiation reaching the radiation detector 13 decreases due to the structure. In this case, the signal value of each pixel of the image data changes according to the structure of the subject M. The signal value includes a pixel value, a density value, and the like. The reading control apparatus 14 outputs the acquired image data of the medical image to the imaging control apparatus 2. Examples of the image reading conditions include a frame rate, a frame interval, a pixel size, and an image size. The frame rate is the number of frames acquired per second, and matches the pulse rate. The frame interval is the time from the start of the operation of acquiring one frame image to the start of the operation of acquiring the next frame image, and matches the pulse interval.

### Configuration Example of Imaging Control Apparatus 2

Next, the imaging control apparatus 2 will be described in detail. As shown in FIG. 1, the imaging control apparatus 2 is constituted by, for example, a computer such as a personal computer or a workstation. The imaging control apparatus 2 includes a controller 21, a storage section 22, an operation part 23, a display part 24, a communication section 25, and an inference section 26. The controller 21, the storage section 22, the operation part 23, the display part 24, the communication section 25, and the inference section 26 are connected to each other by wiring such as a bus 27.

The controller 21 includes at least one processor such as a CPU, a RAM, and the like. CPU is an abbreviation for Central Processing Unit. RAM is an abbreviation for Random Access Memory. The controller 21 reads programs 22a such as a system program and various process programs stored in the storage section 22, loads the programs 22a into the RAM, and executes various processes in accordance with the loaded programs 22a. The controller 21 controls the operation of each component of the imaging control apparatus 2. In the first embodiment, the controller 21 functions as a first acquisition section, a second acquisition section, an image determination section, and the like. Note that the first acquisition section corresponds to a first acquisition step to be described later, the second acquisition section corresponds to a second acquisition step to be described later, and the image determination section corresponds to an image determination step to be described later, and thus detailed description thereof will be given later.

The storage section 22 is a nonvolatile semiconductor memory, a hard disk, or the like. The storage section 22 stores the various programs 22a to be executed by the controller 21, parameters required for execution of processes according to the programs 22a, data of processing results, and the like. The various programs 22a are stored in the form of readable program codes. For example, a program 22a can execute an imaging control step, a first acquisition step, an image processing and adjustment step, a diagnosis support information generation step, a second acquisition step, an image determination step, a display step, and the like. In the imaging control step, control for capturing a medical image is performed. In the first acquisition step, a medical image of the subject M captured by the imaging apparatus 1 is acquired. In the image processing and adjustment step, image processing and image adjustment are performed on the medical image acquired in the first acquisition step. Hereinafter, image processing and image adjustment may be collectively referred to as image adjustment processing or the like. In the diagnosis support information generation step, diagnosis support information is generated by computer processing on the basis of the medical image acquired in the first acquisition step. The computer processing is, for example, inferring and identifying a lesion included in a medical image by using a trained model trained by a neural network or the like. The diagnosis support information is information for supporting a user such as the radiological technologist or an X-ray interpreter to prevent oversight of a lesion. The diagnosis support information is, for example, information for highlighting an inferred and identified lesion region in a medical image. In addition, the diagnosis support information may be, for example, information for notifying whether or not the inferred and identified lesion region exists in the medical image. The diagnosis support information may be displayed on the medical image or may be displayed outside the display region of the medical image. Alternatively, the information may be notified separately from the medical image. In the second acquisition step, the diagnosis support information generated by computer processing on the medical image is acquired. In the image determination step, it is determined to transmit the medical image acquired in the first acquisition step to the image inspection apparatus 3 or the like. For example, in a case where the medical image is acquired by imaging an appropriate region in an appropriate form, or the like, the transmission to the image inspection apparatus 3 is determined. In the display step, the medical image and the diagnosis support information are displayed on the display part 24 before the determination in the image determination step.

The operation part 23 includes at least one of a keyboard, a mouse, a switch, a button, a sensor, and the like. The operation part 23 may be a touch screen combined with the display screen of the display part 24. The operation part 23 accepts/receives various instructions made by user's input operations, and outputs instruction signals corresponding to the accepted instructions to the controller 21.

The display part 24 is a display device such as a liquid crystal display or an organic electro luminescence (EL) display. The display part 24 displays a graphical user interface (GUI) or the like for receiving various input operations from a user such as the radiological technologist in accordance with an instruction of a display signal input from the controller 21. The display part 24 displays the medical image acquired from the imaging apparatus 1 and the diagnosis support information indicating the lesion included in the medical image, before the image determination section of the controller 21 determines to transmit the medical image to the image inspection apparatus 3.

The communication section 25 includes a LAN adapter and a modem. The communication section 25 transmits and receives medical images, various data such as imaging conditions, various signals, and the like to and from the imaging apparatus 1, the image inspection apparatus 3, the image management apparatus 4, and the like connected to the network N.

The inference section 26 includes at least one processor such as a CPU or a GPU, and a memory such as a RAM. GPU is an abbreviation for Graphics Processing Unit. The inference section 26 analyzes and infers a lesion included in the medical image by using the medical image, which has been acquired from the imaging apparatus 1. Specifically, the inference section 26 includes a trained model trained to output an inference result regarding a lesion in response to an input of a medical image. The trained model is stored in, for example, a memory (not illustrated) of the inference section 26 or the storage section 22. The inference section 26 inputs the acquired medical image to the trained model, and analyzes and infers a lesion in the medical image based on output data regarding the lesion output from the trained model. The inference section 26 outputs output data that is an inference result to the controller 21. The output data includes diagnosis support information indicating the detection result of the lesion. Note that the inference function of the inference section 26 may be provided in the controller 21 or in an information processing apparatus different from the imaging control apparatus 2.

Hereinafter, a flow and the like in a case where the above-described trained model is created will be described. For example, a training apparatus including a computer or the like can be used to create the trained model. The training apparatus includes one or more processors such as a CPU(s) and/or a GPU(s), a memory that stores programs and the like, an operation part, a display part, a communication section, and the like. The training apparatus generates, using a pair of a medical image and a ground truth label as training data, a trained model trained to output diagnosis support information on a lesion in response to input of the medical image. Specifically, the training apparatus compares the output data output by the trained model of the training target in response to the input of the medical image for training with the ground truth label, and updates the parameter of the trained model on the basis of an error as the comparison result. For example, in a case where the trained model is implemented by a convolutional neural network, the training apparatus may adjust the parameter of the trained model in accordance with an error between the output result and the correct data in accordance with backpropagation until a predetermined completion condition is satisfied.

### Configuration Example of Image Inspection Apparatus 3

Next, the image inspection apparatus 3 will be described in detail. As shown in FIG. 1, the image inspection apparatus 3 is constituted by, for example, a computer such as a personal computer. The image inspection apparatus 3 includes a controller 31, a storage section 32, an operation part 33, a display part 34, and a communication section 35. The controller 31, the storage section 32, the operation part 33, the display part 34, and the communication section 35 are connected to each other by wiring such as a bus 36.

The controller 31 includes at least one processor such as a CPU, a RAM, and the like. The controller 31 reads programs 32a such as a system program and various process programs stored in the storage section 32, loads the programs 32a into the RAM, and executes various processes in accordance with the loaded programs 32a. The controller 31 controls the operation of each component of the image inspection apparatus 3. In the first embodiment, the controller 31 functions as a diagnostic image determination section and the like. Note that the diagnostic image determination section corresponds to a diagnostic image determination step to be described later, and therefore detailed description thereof will be given later.

The storage section 32 is a nonvolatile semiconductor memory, a hard disk, or the like. The storage section 32 stores the various programs 32a to be executed by the controller 31, parameters required for execution of processes according to the programs 32a, data of processing results, and the like. The various programs 32a are stored in the form of readable program codes. For example, a program 32a is capable of executing a first reception step, a second reception step, a display step, an image inspection and adjustment step, a diagnostic image determination step and the like. In the first reception step, the communication section 35 is controlled to receive a medical image from the imaging control apparatus 2. In the second reception step, the communication section 35 is controlled to receive diagnosis support information from the imaging control apparatus 2. In the display step, the medical image and the diagnosis support information are displayed on the display part 34 before the determination in the diagnostic image determination step. In the image inspection and adjustment step, image inspection and adjustment are performed on the medical image received in the first reception step. In the diagnostic image confirmation step, it is determined to transmit the medical image or the like acquired in the first reception step to the image management apparatus 4.

The operation part 33 includes at least one of a keyboard, a mouse, a switch, a button, a sensor, and the like. The operation part 33 may be a touch screen combined with the display screen of the display part 34. The operation part 33 accepts/receives various instructions made by user's input operations, and outputs instruction signals corresponding to the accepted instructions to the controller 31.

The display part 34 is a display device such as a liquid crystal display or an organic EL display. The display part 34 displays a GUI for receiving various input operations from the radiological technologist or the like, an image inspection screen for determining whether an image inspection result is pass, and the like in accordance with instructions of display signals input from the controller 31. On the image inspection screen, for example, the medical image acquired from the imaging control apparatus 2, the diagnosis support information indicating a lesion included in the medical image, and the like are displayed.

The communication section 35 includes a LAN adapter and a modem. The communication section 35 transmits and receives various data such as medical images and various signals to and from the imaging control apparatus 2, the image management apparatus 4, and the like connected to the network N. In the first embodiment, the communication section 35 also functions as part of the above-described first receiver and the second receiver. The first receiver receives the medical image from the imaging control apparatus 2. The second receiver receives the diagnosis support information on a lesion included in the medical image from the imaging control apparatus 2.

### Operation Example of Image Capturing System 100A

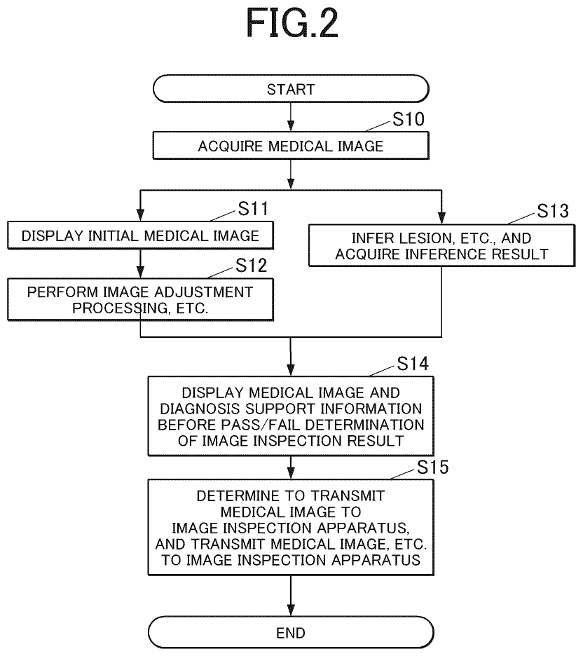

In the first embodiment, it is determined in the imaging control apparatus 2 whether a medical image has been obtained by imaging an appropriate region in an appropriate form, and it is determined in the image inspection apparatus 3 whether the image inspection result of the medical image is pass or not (fail). FIG. 2 is a flowchart illustrating an example of the operation of the imaging control apparatus 2 in a case where a medical image is transmitted to the image inspection apparatus 3 according to the first embodiment. The controller 21 implements an imaging control step, a first acquisition step, an image processing and adjustment step, a diagnosis support information generation step, a second acquisition step, an image determination step, a display step, and the like by executing a program 22a.

As illustrated in FIG. 2, the controller 21 acquires a medical image of a predetermined imaging part of a subject captured by the imaging apparatus 1 (Step S10). In the present embodiment, a case where the imaging part is a chest will be described. Note that the imaging part is not limited to the chest but may be another part (abdomen, foot, or the like), and the imaging direction is not limited to the front but may be a side or the like.

The controller 21 allows the display part 24 to display the acquired medical image as an initial medical image on the imaging screen (Step S11). The initial medical image in this case is a medical image before the predetermined image adjustment processing or the like is performed.

The controller 21 executes predetermined image adjustment processing or the like on the acquired medical image (Step S12). For example, gradation processing for adjusting the density of a medical image, rotation processing of a medical image, horizontal inversion of a medical image, black-and-white inversion processing, other emphasis processing, and the like are exemplified. The radiological technologist or the like can determine whether or not the initial medical image displayed on the imaging screen has been obtained by imaging an appropriate region in an appropriate form. That is, the radiological technologist or the like can view the initial medical image and make a primary determination as to whether re-imaging is necessary or not. In this case, the radiological technologist or the like may determine the presence or absence of imaging failure, for example, whether the target part is fully in the imaging area or whether a body motion or the like of the subject did not occur. Note that the determination of whether or not the initial medical image is an image of an appropriate region in an appropriate form may be performed by the controller 21 instead of the user.

The inference section 26 executes inference of a lesion included in the medical image in parallel with the image adjustment processing or the like of the medical image by the controller 21, and acquires the inference result (Step S13). When the imaging part is the chest, examples of the lesion that can be detected include a nodule shadow, a tumor shadow, an infiltrative shadow, and pneumothorax. Specifically, the inference section 26 inputs the medical image acquired from the imaging apparatus 1 to the trained model, and acquires the output data output from the trained model. Based on the acquired output data, the inference section 26 infers and identifies a lesion included in the medical image of the imaging part. The inference section 26 generates and acquires diagnosis support information for supporting, for example, prevention of oversight of a lesion, on the basis of the inference result of the lesion, and outputs the diagnosis support information to the controller 21. Note that if the medical image does not include a lesion, the inference section 26 may output, to the controller 21, information indicating that the medical image is normal without a lesion as output data.

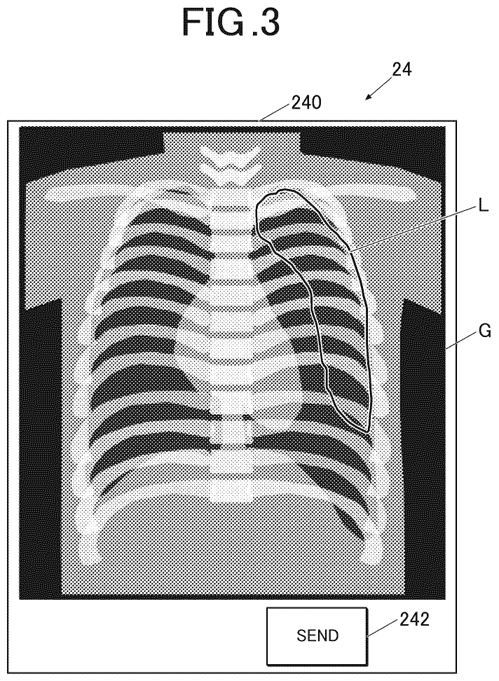

The controller 21 displays the medical image and the diagnosis support information indicating the lesion on the imaging screen of the display part 24 before the transmission of the medical image to the image inspection apparatus 3 is determined, that is, before it is determined in the image inspection apparatus 3 whether or not the image inspection result is pass (Step S14). FIG. 3 is a diagram illustrating a medical image G and diagnosis support information L displayed on the imaging screen 240 of the display part 24 according to the first embodiment. For example, when the lesion is pneumothorax and the pneumothorax is found in the left lung, the diagnosis support information L indicating that the lesion has been detected in the left lung of the chest is superimposed on the medical image G and displayed. The diagnosis support information L includes, for example, a substantially elliptical or substantially polygonal frame surrounding an air region formed between the left lung and the chest cavity. Note that the diagnosis support information L is not limited to the frame, and the lesion region may be highlighted in a predetermined color. In addition, a send button 242 for determining to transmit the medical image G and the diagnosis support information L being displayed to the image inspection apparatus 3 is provided on an imaging screen 240 of the display part 24.

The radiological technologist or the like can check whether or not there is a lesion with a high degree of urgency, such as pneumothorax, from the medical image G and the diagnosis support information L displayed on the imaging screen 240 before pass/fail determination of the image inspection result in the image inspection apparatus 3. In a case where a lesion such as pneumothorax is found, the radiological technologist or the like can immediately notify a doctor such as a clinician or an X-ray interpreter of the lesion result as necessary. Thus, appropriate treatment can be performed on the patient on the spot. Examples of the means for contacting the doctor include a mobile phone, a smartphone, an email, and an SNS. On the other hand, when the radiological technologist or the like determines that there is no lesion with a high degree of urgency such as pneumothorax and determines that the medical image G has been obtained by imaging an appropriate region in an appropriate form, the radiological technologist or the like determines to transmit the medical image G to the image inspection apparatus 3. In this case, the radiological technologist or the like selects the send button 242 provided on the imaging screen of the display part 24 by operating the operation part 23.

When acquiring an instruction to transmit the medical image to the image inspection apparatus 3, the controller 21 transmits inspection data including the medical image and the diagnosis support information to the image inspection apparatus 3 (Step S15). For example, when acquiring an instruction corresponding to a selection operation on the send button 242 made by the radiological technologist or the like on the imaging screen, the controller 21 determines to transmit the medical image or the like to the image inspection apparatus 3. In this case, the controller 21 transmits the inspection data including the medical image and the like subjected to the image adjustment processing or the like to the image inspection apparatus 3 via the network N.

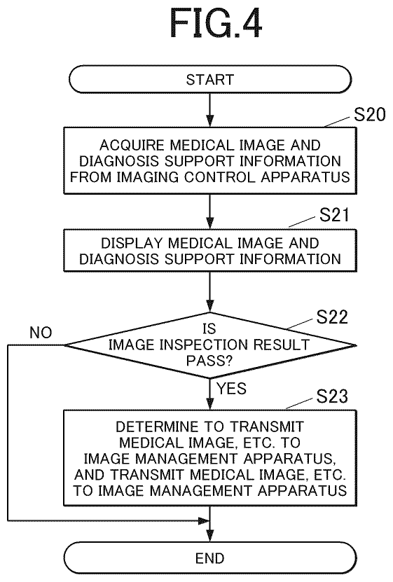

FIG. 4 is a flowchart illustrating an example of the operation of the image inspection apparatus 3 in a case of determining whether an image inspection result of a medical image or the like is pass or fail according to the first embodiment. The controller 31 of the image inspection apparatus 3 realizes various processes including a first reception step, a second reception step, an image determination step, and the like by executing a program 32a stored in the storage section 32 or the like.

As illustrated in FIG. 4, the communication section 35 receives inspection data including a medical image of a predetermined patient and diagnosis support information thereon from the imaging control apparatus 2. The controller 31 acquires the inspection data including the medical image and the diagnosis support information on the predetermined patient received by the communication section 35 (Step S20).

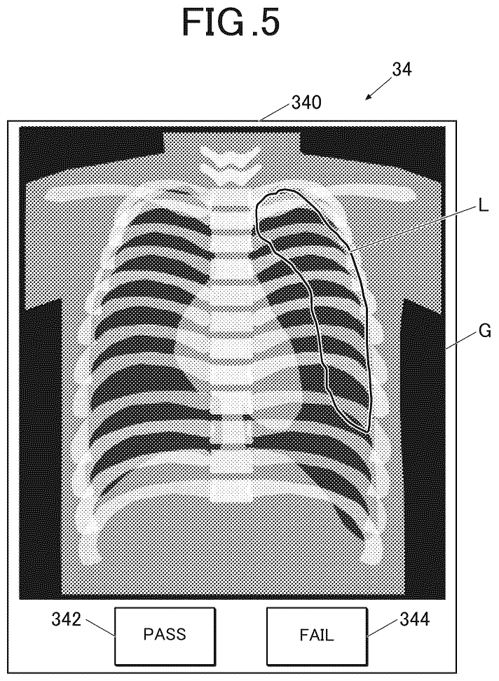

Based on the acquired inspection data, the controller 31 causes the display part 34 to display the medical image and the diagnosis support information on the image inspection screen (Step S21). FIG. 5 is a diagram showing the medical image G and the diagnosis support information L displayed on an image inspection screen 340 of the display part 34 according to the first embodiment. Note that the displayed content is substantially the same as the displayed content in FIG. 3 described above, and thus the detailed description of the common content will be omitted. The medical image G and the diagnosis support information L superimposed on the medical image G are displayed on the image inspection screen 340. Under the medical image G, a pass button 342 is provided for determining to transmit the medical image G and the like to the external image management apparatus 4 when the image inspection result is pass. At a position adjacent to the pass button 342, a fail button 344 is provided for determining not to transmit the medical image G and the like to the external image management apparatus 4 when the image inspection result is fail. Note that in the present embodiment, the button to be selected when the image inspection result is pass is set as the pass button 342, but the button name may be “image output button” or “inspection end button”. Furthermore, a configuration may be adopted in which, in a case where it is not determined to transmit the medical image G and the like to the external image management apparatus 4 within a predetermined time, it is determined that the image inspection result is fail, and it is determined not to transmit the medical image G and the like to the external image management apparatus 4. In this case, the fail button 344 may be omitted.

The controller 31 determines whether the image inspection result of the medical image is pass (Step S22). For example, the radiological technologist or the like makes a secondary determination as to whether the medical image being displayed on the display part 34 is an image suitable for diagnosis. That is, the radiological technologist or the like determines whether the medical image displayed on the display part 34 is the optimal medical image for the clinician or the like to make a diagnosis after the medical image is transmitted to the image management apparatus 4. For example, in a case where the density of the medical image is appropriate, it is determined that the medical image is an image suitable for diagnosis. Furthermore, for example, in a case where the image is at the same angle of view as the past image, it is determined that the medical image is an image suitable for diagnosis. If the density of the medical image is significantly inappropriate, gradation processing may be performed on the medical image. Furthermore, in a case where the medical image is not at the same angle of view as the past image, enlargement or reduction processing may be performed on the medical image. The controller 31 performs image inspection and adjustment such as gradation processing, enlargement processing, or reduction processing on the medical image on the basis of an instruction from the radiological technologist or the like. When the radiological technologist or the like determines that the medical image is an image suitable for diagnosis and the image inspection result is pass, the radiological technologist or the like selects the pass button 342 on the image inspection screen 340. The determination of whether to perform the image inspection and adjustment described above may be performed by the controller 31 instead of the radiological technologist or the like.

When determination is made that the inspection result of the medical image is pass, the controller 31 proceeds to Step S23. For example, the controller 31 determines that the image inspection result of the medical image is pass when the pass information corresponding to the selection of the pass button 342 is acquired on the image inspection screen 340. In this case, the controller 31 determines to transmit the inspection data including the medical image and the diagnosis support information to the image management apparatus 4, and transmits the inspection data to the image management apparatus 4 (Step S23). If the medical image has been subjected to the image inspection and adjustment, the controller 31 transmits the medical image subjected to the image inspection and adjustment to the image management apparatus 4. The image management apparatus 4 receives the inspection data from the inspection apparatus 3, and stores and manages the medical image and the diagnosis support information of the predetermined patient included in the received inspection data. Thus, an X-ray interpreter, a clinician, or the like can see a medical image, diagnosis support information, and the like of a predetermined patient by accessing the image management apparatus 4 from a client terminal or the like.

On the other hand, when acquiring fail information corresponding to the selection of the fail button 344 on the image inspection screen 340, the controller 31 determines that the image inspection result of the medical image is fail. In this case, the controller 31 determines not to transmit the inspection data including the medical image and the diagnosis support information to the image management apparatus 4. Therefore, the controller 31 ends the series of processes without transmitting the inspection data including the medical image and the like to the image management apparatus 4. Further, the radiological technologist or the like on the imaging control apparatus 2 side may be notified that the image inspection result is fail, and may be requested to re-capture a medical image (re-imaging). Examples of the instruction means to the radiological technologist or the like include a mobile phone, a smartphone, an e-mail, and an SNS. Furthermore, the image inspection screen of the display part 34 may be provided with a re-imaging button (image-rejected button) for notifying the radiological technologist or the like on the imaging control apparatus 2 side of that the image is rejected (imaging failure) and for requesting re-imaging. Alternatively, the fail button 344 may also serve as a re-imaging button (image-rejected button).

According to the first embodiment, the following effects can be achieved. That is, conventionally, a medical image or the like is transmitted from the imaging control apparatus 2 to the image inspection apparatus 3, and the image inspection apparatus 3 displays diagnosis support information only for a medical image whose image inspection result is pass. Alternatively, when a medical image whose image inspection result is pass is transmitted to the image management apparatus 4, diagnosis support information is also appended. In these cases, a log period of time may elapse before the doctor interprets the medical image and the diagnosis support information stored in the image management apparatus 4. Therefore, if the doctor determines, at this stage, that there is a finding in the medical image, the patient may have gone home, and there is a problem in that appropriate treatment cannot be immediately performed on the patient. In particular, in the case of a lesion with a high degree of urgency such as pneumothorax, there is a possibility that the patient becomes seriously ill at home or the like.

On the other hand, in the first embodiment, the medical image and the diagnosis support information are displayed on the display part 24 before the imaging control apparatus 2 determines to transmit the medical image to the image inspection apparatus 3, that is, before the pass/fail determination of the image inspection result of the medical image in the image inspection apparatus 3. Thus, the radiological technologist or the like can quickly grasp a lesion with a high degree of urgency, such as pneumothorax, by checking the medical image and the diagnosis support information displayed on the imaging screen of the display part 24. As a result, the radiological technologist or the like can immediately notify a doctor such as an X-ray interpreter of the lesion as necessary. In other words, before the pass/fail determination of the image inspection result of the medical image in the image inspection apparatus 3, the STAT image report in which the radiological technologist or the like informs the X-ray interpreter or the like of the findings of the captured medical image can be made. In addition, appropriate treatment can be promptly administered to the patient on the spot, and it is possible to prevent the patient from becoming seriously ill due to a delay in diagnosis or a lesion being overlooked. Note that an example of the appropriate treatment includes drainage.

### Second Embodiment

The second embodiment is different from the first embodiment in that it is determined in the imaging control apparatus 2 whether or not the image inspection result of the medical image is pass. Note that differences from the first embodiment will be mainly described, constituent elements substantially the same as those of the first embodiment will be denoted by the same reference signs, and their descriptions will be omitted or simplified.

### Configuration Example of Image Capturing System 100B

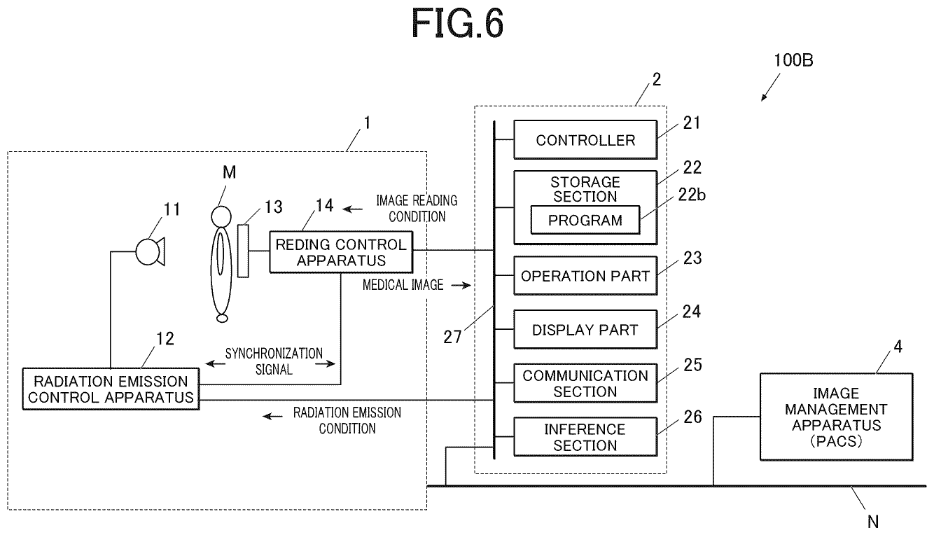

FIG. 6 is a diagram illustrating an example of a schematic configuration of an image capturing system 100B according to the second embodiment. The image capturing system 100B corresponds to an example of a medical system and includes an imaging apparatus 1, an imaging control apparatus 2, an image management apparatus 4, and the like. The imaging apparatus 1, the imaging control apparatus 2, and the image management apparatus 4 are communicably connected to each other via a network N such as a LAN, the Internet, or a WAN. A communication method of the network N may be wired communication or wireless communication. In the second embodiment, the imaging control apparatus 2 has the function of the image inspection apparatus 3 described in the first embodiment. That is, the imaging control apparatus 2 and the image inspection apparatus 3 are integrally formed.

As shown in FIG. 6, the imaging control apparatus 2 is constituted by, for example, a computer such as a personal computer. The imaging control apparatus 2 includes a controller 21, a storage section 22, an operation part 23, a display part 24, a communication section 25, and an inference section 26. The controller 21, the storage section 22, the operation part 23, the display part 24, the communication section 25, and the inference section 26 are connected to each other by wiring such as a bus 27. The imaging control apparatus 2 has a function of determining whether the medical image captured by the imaging apparatus 1 is an image of an appropriate region in an appropriate form, and a function of determining whether the medical image captured by the imaging apparatus 1 is an image suitable for diagnosis, that is, whether the image inspection result of the medical image is pass. The imaging control apparatus 2 functions as a first receiver, a second receiver, an image inspection and adjustment section, and a diagnostic image determination section. Note that the first receiver corresponds to a first reception step to be described later, the second receiver corresponds to a second reception step to be described later, the image inspection and adjustment section corresponds to an image inspection and adjustment step to be described later, and the diagnostic image determination section corresponds to a diagnostic image determination step to be described later.

The storage section 22 stores various programs 22a and the like to be executed by the controller 21. For example, a program 22a can execute an imaging control step, a first acquisition step, an image processing and adjustment step, a diagnosis support information generation step, a second acquisition step, a display step, a diagnostic image determination step, and the like. In the diagnostic image determination step, for example, when a medical image is an image suitable for diagnosis, that is, when the image inspection result of the medical image is pass, the transmission to the image management apparatus 4 is determined.

### Operation Example of Imaging Control Apparatus 2

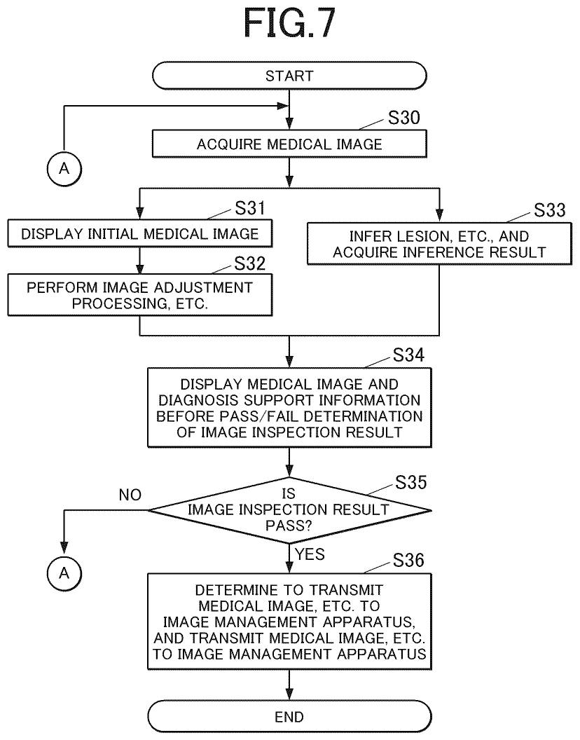

FIG. 7 is a flowchart illustrating an example of the operation of the imaging control apparatus 2 in a case where a medical image is transmitted to the image management apparatus 4 according to the second embodiment. The controller 21 of the imaging control apparatus 2 executes a program 22a to realize an imaging control step, a first acquisition step, an image processing and adjustment step, a diagnosis support information generation step, a second acquisition step, a display step, an image determination step, and the like.

The controller 21 acquires a medical image of the imaging part of the subject captured by the imaging apparatus 1 (Step S30). The imaging part is the front of the chest, as in the first embodiment. Next, the controller 21 displays the acquired medical image as the initial medical image on the imaging screen of the display part 24 (Step S31). The initial medical image in this case is a medical image before predetermined image processing is performed.

The controller 21 executes predetermined image adjustment processing and the like on the acquired medical image (Step S32). The radiological technologist or the like can determine whether or not the initial medical image displayed on the imaging screen has been obtained by imaging an appropriate region in an appropriate form. Note that the determination of whether or not the initial medical image is an image of an appropriate region in an appropriate form may be performed by the controller 21 instead of the user.

The inference section 26 executes inference of a lesion included in the medical image in parallel with the image adjustment processing or the like of the medical image by the controller 21, and acquires the inference result (Step S33). The inference section 26 inputs the medical image acquired from the imaging apparatus 1 to the trained model, and acquires output data output from the trained model. The inference section 26 infers and identifies a lesion included in the medical image based on the acquired output data. The inference section 26 generates diagnosis support information for supporting, for example, prevention of oversight of a lesion on the basis of an inference result of the lesion, and outputs the diagnosis support information to the controller 21. Note that if the medical image does not include a lesion, the inference section 26 may output, to the controller 21, information indicating that the medical image is normal without a lesion as output data.

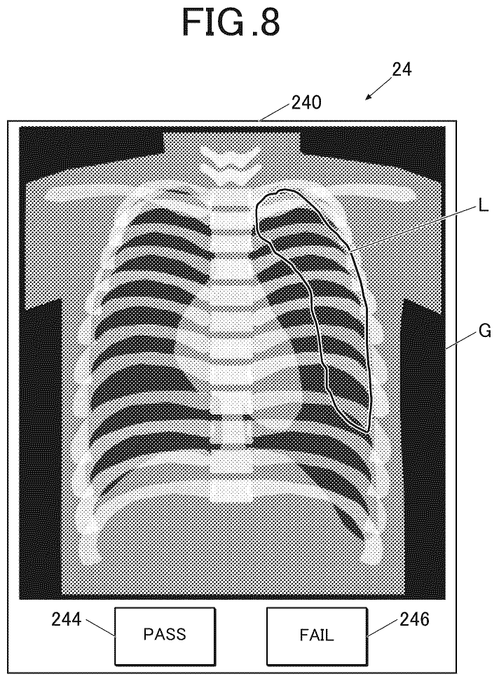

The controller 21 displays the medical image and the diagnosis support information indicating the lesion on the imaging screen of the display part 24 before the transmission of the medical image to the image management apparatus 4 is determined, that is, before it is determined whether or not the image inspection result is pass (Step S34). FIG. 8 is a diagram illustrating the medical image G and the diagnosis support information L displayed on the imaging screen 240 of the display part 24 according to the second embodiment. For example, when the lesion is pneumothorax and the pneumothorax is found in the left lung, the diagnosis support information L indicating that the lesion has been detected in the left lung of the chest is superimposed on the medical image G and displayed. Under the medical image G, the pass button 244 is provided for determining that the medical image G or the like is to be transmitted to the external image management apparatus 4 when the image inspection result is pass. At a position adjacent to the pass button 244, a fail button 246 for determining that the medical image G or the like is not transmitted to the external image management apparatus 4 when the image inspection result is fail is provided.

The controller 21 determines whether the image inspection result of the medical image is pass (Step S35). For example, the radiological technologist or the like determines whether the medical image displayed on the display part 24 is an image suitable for diagnosis. If the density of the medical image is significantly inappropriate, gradation processing may be performed on the medical image. Furthermore, in a case where the medical image is not at the same angle of view as the past image, enlargement or reduction processing may be performed on the medical image. The controller 21 performs image inspection and adjustment such as gradation processing, enlargement/reduction processing, or the like on the medical image based on an instruction from the radiological technologist or the like. When the radiological technologist or the like determines that the medical image is suitable for diagnosis and the image inspection result is pass, the radiological technologist or the like selects the pass button 244 on the image inspection screen. Note that the controller 21 may determine whether to execute the above-described image adjustment processing, instead of the radiological technologist or the like.

When determination has been made that the medical image inspection result is pass, the controller 21 proceeds to Step S36. For example, when acquiring pass information corresponding to selection of the pass button 244 on the imaging screen 240, the controller 21 determines that the image inspection result of the medical image is pass. In this case, upon acquiring the pass information, the controller 21 determines to transmit the inspection data including the medical image and the diagnosis support information to the image management apparatus 4, and transmits the inspection data to the image management apparatus 4 (Step S36). In the case where the medical image G has been subjected to the image inspection and adjustment, the controller 21 transmits the medical image G subjected to the image inspection and adjustment to the image management apparatus 4.

On the other hand, when acquiring fail information corresponding to selection of the fail button 246 on the image inspection screen 240, the controller 21 determines that the image inspection result of the medical image is fail. In this case, the controller 21 determines not to transmit the inspection data including the medical image and the diagnosis support information to the image management apparatus 4. Therefore, the controller 21 ends the series of processes without transmitting the inspection data including the medical image and the like to the image management apparatus 4. In this case, the radiological technologist or the like may re-capture a medical image.

According to the second embodiment, substantially the same effects as those of the first embodiment described above can be achieved. Before the imaging control apparatus 2 determines to transmit the medical image to the image management apparatus 4, that is, before the pass/fail determination of the image inspection result of the medical image, the medical image and the diagnosis support information are displayed on the display part 24. Thus, the radiological technologist or the like can quickly grasp a lesion with a high degree of urgency, such as pneumothorax, by checking the medical image and the diagnosis support information displayed on the imaging screen of the display part 24. As a result, the radiological technologist or the like can immediately notify a doctor such as an X-ray interpreter of the lesion as necessary, so that an appropriate treatment can be applied to the patient on the spot, and it is possible to prevent the patient from becoming seriously ill due to a delay in diagnosis or an oversight of a lesion. In addition, according to the second embodiment, since the imaging control apparatus 2 has the function of the image inspection apparatus 3, the radiological technologist or the like can more quickly determine whether the image inspection result of the medical image is pass or not.

Although some preferred embodiments of the present disclosure have been described in detail with reference to the accompanying drawings, the technical scope of the present disclosure is not limited to these examples. Furthermore, those to which various changes/modifications and improvements have been applied naturally belong to the technical scope of the present disclosure within the range of the technical idea described in claims.

For example, in the first embodiment, when performing dynamic analysis or the like on a medical image, whether or not the image state is suitable for dynamic analysis processing or the like required for diagnosis may be determined in the step of image adjustment processing or the like and the step of determining whether the image inspection result is pass or not. In the second embodiment too, in the step of the image adjustment processing or the like and the step of determining whether or not the image inspection result is pass, it may be determined whether or not the image state is suitable for the dynamic analysis processing or the like required for diagnosis.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. A non-transitory computer-readable storage medium storing a program causing a computer to:
acquire a medical image in which a subject is imaged;
acquire diagnosis support information generated by computer processing on the medical image;
determine to transmit the acquired medical image to an external apparatus; and
display the medical image and the diagnosis support information before the determining.

2. The non-transitory computer-readable storage medium according to claim 1,
wherein the program further causes the computer to:
perform control to capture the medical image; and
perform image processing and image adjustment on the acquired medical image, and
wherein the determining includes determining to transmit the medical image subjected to the image processing and the image adjustment to the external apparatus.

3. The non-transitory computer-readable storage medium according to claim 1,
wherein the program further causes the computer to generate the diagnosis support information by the computer processing based on the acquired medical image, and
wherein the acquiring of the diagnosis support information includes acquiring the generated diagnosis support information.

4. The non-transitory computer-readable storage medium according to claim 3, wherein the diagnosis support information is a lesion detection result.

5. The non-transitory computer-readable storage medium according to claim 1,
wherein the acquiring of the medical image includes receiving the medical image from an imaging control apparatus,
wherein the acquiring of the diagnosis support information includes receiving the diagnosis support information from the imaging control apparatus,
wherein the determining includes determining to transmit the received medical image to an image management apparatus, and
wherein the displaying includes displaying the medical image and the diagnosis support information before the determining.

6. The non-transitory computer-readable storage medium according to claim 5,
wherein the program further causes the computer to perform image processing and image adjustment on the received medical image,
wherein the performing of the image processing and the image adjustment includes performing image inspection and adjustment on the received medical image, and
wherein the determining includes determining to transmit the medical image subjected to the image inspection and the adjustment to the image management apparatus.

7. An imaging control apparatus comprising a hardware processor that
acquires a medical image in which a subject is imaged,
acquires diagnosis support information generated by computer processing on the medical image,
determines to transmit the acquired medical image to an external apparatus, and
displays the medical image and the diagnosis support information before the determining.

8. A medical system comprising:
the imaging control apparatus according to claim 7; and
an image inspection apparatus that
receives the medical image from the imaging control apparatus,
receives the diagnosis support information from the imaging control apparatus, and
determines to transmit the medical image to an image management apparatus.

9. The medical system according to claim 8, wherein the image inspection apparatus
performs image inspection and adjustment on the medical image, and
determines to transmit the medical image subjected to the image inspection and the adjustment to the image management apparatus.
